(19) United States
(12) Patent Application Publication
(10) Pub. No.: US 2008/0249088 A1
(43) Pub. Date: Oct. 9, 2008

(54) Topical Delivery of Antifungal Drugs

(76) Inventor: William L. Sherrett, Milwaukie, OR (US)

Correspondence Address: William L Sherrett, Apt 122, 12205 SE 31st PI, Milwaukie, OR, 97222, US

(21) Appl. No.: 11/733,076

(22) Filed: Apr. 9, 2007

(51) Int. Cl. A61K 31/535; A61K 31/417; A61P 17/00; A61K 31/13

(52) U.S. Cl. 514/231.2; 514/396; 514/462; 514/657

(57) ABSTRACT

A topical formula for treatment of antifungal infections of the skin and nails comprises: (a) a therapeutically effective amount of a benzylamine or morpholine antifungal compound; (b) an aliphatic alcohol substituted with an aromatic substituent in which the antifungal compound is soluble to a degree that a therapeutically effective concentration of the antifungal compound can be applied topically in solution; and (c) a distribution solvent or mixture of solvents in which the solvent mixture of parts (a) and (b) is soluble, and which has a boiling point that is less than about 110.degree.C. The benzylamine compound can be butenafine. The morpholine compound can be amorolfine. The aliphatic alcohol substituted with an aromatic substituent can be benzyl alcohol, or phenethyl alcohol. The distribution solvent mixture can include ethyl alcohol, water, or isopropyl alcohol. Alternatively, the formula can include a second antifungal compound.

### CROSS REFERENCE TO RELATED APPLICATIONS

Not Applicable

### BACKGROUND OF THE INVENTION

The present invention generally relates to a method of delivery of drugs through topical application to the skin and nails, especially some of the newer antifungal compounds.

Dermatophyte infections of the skin and nails, caused by several species of fungi, have demonstrated a remarkable resilience against the growing array of antifungal agents developed to eliminate them. Conventional wisdom in the medical community holds that once an infection has become established in a substantial area of skin or nails, it may not be possible to remove it permanently with either local or systemic treatments.

Systemic treatments with newer antifungals, such as terbinafine, have shown effectiveness against nail infections, but at great cost over the long periods needed for effective treatment, along with potential for side effects. The same systemic treatments have shown limited effectiveness against chronic skin infections, even when combined with the best of current topical treatments. Since clearing a nail infection is pointless unless the adjacent skin is also cleared of infection, the need for a more potent topical antifungal treatment for skin is underscored.

The epidermis is composed of an outer layer of keratinized cells interwoven with lipids, and inner layers of viable cells in an aqueous suspension. Antifungal compounds are generally hydrophobic, so a means is needed to carry them through the lipid environment first, then into the aqueous environment. Current topical formulas mainly address the lipid solubility, thus failing to effect adequate penetration into the deeper strata of the epidermis.

A predictable result of widespread and long-term under treatment of these skin infections is the emergence of strains that have shown resistance to the antifungal agents used. By failing to deliver the antifungal drugs deeply enough into the skin to eliminate the infection, and requiring reapplication with each recurrence of symptoms, the current topical formulas have only compounded the problem of treatment.

One approach has shown potential, however.

U.S. Pat. No. 4,820,724 describes a dual-phase solvent system conceived for griseofulvin that uses a high boiling-point alcohol to carry the active ingredient into the skin, after being distributed onto the surface of the skin in a thin layer by a compatible low boiling-point solvent. The griseofulvin formula proved to be effective on skin, but not on nails.

U.S. Pat. App. 20050238672 describes an improvement on the above mentioned system which adds water to the alcohol mixture, and uses terbinafine as the preferred active ingredient at a 2% concentration.

One key advantage of these formulas is that the active ingredient is suspended by the high boiling-point alcohol in a state that is soluble in both lipids and water. The addition of water to the solvent mix in the improved formula is intended for enhanced penetration of the nail plate.

A shortcoming of much of the current research in this field lies in the primary focus on the nail infection problem. Clearing a skin and nail infection is necessarily a two-stage process, with the skin as the first stage and the nail as the second stage. Only recently have new formulas appeared on the market which claim to cure skin infections permanently with treatment regimens of from one day to one week, and no studies of the efficacy of these formulas have extended beyond eight weeks duration. Thus, there remains a need for an effective topical treatment for skin infections to work in concert with a nail treatment, which can use the current antifungal agents to their full potential.

Combining the dual-phase formula with some of the best of the antifungals available today, such as the benzylamine derivative butenafine, or the mopholine derivative amorolfine, might provide for a near ideal topical formula for infected skin, and might well also clear nail infections. The preferred formula combines the anti-itch properties of benzyl alcohol with the anti-inflammatory properties of butenafine. The higher potency of butenafine vs. terbinafine against dermatophytes allows for a concentration of 1% in the preferred embodiment.

### SUMMARY OF THE INVENTION

The invention is directed to combining a solvent carrier system to one or more active ingredients chosen from currently available antifungal agents for the purpose of providing a superior topical treatment of fungal infections of the skin and nail.

The solvent system comprises a first solvent phase of a high boiling-point alcohol in which the active ingredient is soluble, and a second solvent phase of a solvent or mixture of solvents in which the above mixture is soluble, and which also has a boiling point less than 110.degree.C.

The primary active ingredient is chosen from the group consisting of benzylamine or morpholine derivatives. The composition of the above invention may be applied to the skin by aerosol, pump-spray bottle, squeeze bottle, brush, or any other means of applying a topical liquid.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

One embodiment of the present invention is an antifungal composition for topical application to the skin and nails. In general, this embodiment of the composition comprises:

(a) a therapeutically effective amount of a benzylamine or a morpholine antifungal compound;

(b) an aliphatic alcohol substituted with an aromatic substituent in which the antifungal compound is soluble to a degree that a therapeutically effective concentration of the antifungal compound can be applied topically in solution;

(c) a distribution solvent or mixture of solvents in which the solvent mixture of parts (a) and (b) is soluble, and which has a boiling point that is less than about 110.degree.C.

Typically, the antifungal compound is butenafine. However, the antifungal compound can be amorolfine, or any analog or derivative of benzylamine or morpholine.

Typically, the aliphatic alcohol substituted with an aromatic substituent is benzyl alcohol. However, in another alternative, the aromatic alcohol is phenethyl alcohol or another aromatic alcohol.

Typically, the distribution solvent or mixture of solvents is selected from the group consisting of a lower aliphatic alcohol in which the solvent mixture of parts (a) and (b) is soluble, water, or a water-compatible solvent mixture. Preferably, the group consists of ethanol and water.

Typically, the concentration of the antifungal compound is from about 0.05% to about 3% (w/v) in the final composition. Preferably, the concentration of the antifungal compound is about 1% (w/v) in the final composition.

Typically, the concentration of the aliphatic alcohol substituted with an aromatic substituent is from about 2% to about 25% (v/v) in the final composition. Preferably, the concentration of the aliphatic alcohol substituted with an aromatic substituent is about 5%.

Typically, the concentration of the distribution solvent or mixture of solvents is about 75% to about 98% (v/v) in the final composition. Preferably, the concentration of the distribution solvent or mixture of solvents is about 95% (v/v).

The invention is primarily aimed at the delivery of drugs from the surface of the skin into the full depth of the epidermal tissue in a concentration sufficient to provide a fungicidal affect. It is intended to be equally effective in the highly keratinized epithelium, such as found in the plantar and peri-plantar regions of the foot and the subungual epithelium surrounding the nail bed, as well as the thinner epithelial layers of the upper thigh. The secondary aim of the invention is the delivery of drugs from the surface of the nail plate into the plate and matrix, and under the nail plate to the nail bed.

In the preferred embodiment, a 100 ml. mixture would be created by mixing 1 gram of butenafine with 5 ml. of benzyl alcohol at room temperature. This would then be dissolved into 85 ml. of ethanol, then 10 ml. of water would be added. Butenafine has limited solubility in water, but experiments have shown that this combination of solvents can retain hydrophobic compounds in solution until the water content reaches 40-60%.

Upon application to the skin, the ethanol and water mix helps to make the skin more permeable, and, as it evaporates away, it distributes the remaining benzyl alcohol and butenafine solution into a thin layer at a 20% concentration. This solution will carry the butenafine throughout the lipid environment of the stratum corneum and also into the viable epidermis, where it will precipitate out when the moisture concentration reaches 40% to 60%.

It is theorized that application of the formula leads to the precipitation of the active agent between the keratin cells in a crystalline form, where it can then act in a slow-release manner. Systemic treatment, by contrast, doesn't seem to result in a high enough concentration throughout the epidermis to effect removal of the infection. Other topical formulas fail to deliver the active ingredient past the lipid environment of the stratum corneum to an effective concentration.

In an alternative embodiment, a second antifungal agent may be added to the formula. In that case, the formula would be adjusted by deducting an equivalent amount from the more volatile alcohol. For example, adding a 1% griseofulvin element to the above embodiment would result in the ethanol being reduced by 1%.

### ADVANTAGES OF THE INVENTION

The present invention provides methods and compositions that are far more efficient than systemic treatments in removing fungal skin infections. This advantage may well apply to nail infections also. The invention has demonstrated superior effectiveness over other topical formulas currently available. Any recurrence of symptoms can be easily treated due to the simplicity and safety of the invention.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

### REFERENCES

Singal A, Pandhi D, Agrawal S, Das S, Comparative efficacy of topical 1% butenafine and 1% clotrimazole in tinea cruris and tinea corporis: a randomized, double-blind trial., J Dermatolog Treat. 2005;16(5-6):331-5
Mukheijee P K, Leidich S D, Isham N, Leitner I, Ryder N S, Ghannoum M A, Clinical Trichophyton rubrum strain exhibiting primary resistance to terbinafine, Antimicrob Agents Chemother. January 2003; 47(1):82-6
Saple D G, Amar A K, Ravichandran G, Korde K M, Desai A, Efficacy and safety of butenafine in superficial dermatophytoses, J Indian Med Assoc. May 2001; 99(5):274-5
U.S. Patent Documents:
4,820,724 April 1989 Nimni

I claim:

1. An antifungal composition for topical application to the skin and nails comprising: (a) a therapeutically effective amount of an antifungal compound selected from the list consisting of benzylamine derivatives and morpholine derivatives; (b) an aliphatic alcohol substituted with an aromatic substituent in which the antifungal compound is soluble to a degree that a therapeutically effective concentration of the antifungal compound can be applied topically in solution; and (c) a distribution solvent or mixture of solvents in which the solvent mixture of parts (a) and (b) is soluble, and which has a boiling point that is less than about 110.degree.C.

2. The antifungal composition of claim 1 wherein the antifungal compound is selected from the list consisting of butenafine, amorolfine, and all analogs and derivatives of butenafine and amorolfine.

3. The antifungal composition of claim 2 wherein the antifungal compound is butenafine.

4. The antifungal composition of claim 2 wherein the aliphatic alcohol is selected from the group consisting of benzyl alcohol and phenethyl alcohol.

5. The antifungal composition of claim 4 wherein the aliphatic alcohol is benzyl alcohol.

6. The antifungal composition of claim 1 wherein the distribution solvent or mixture of solvents comprises one or more lower aliphatic alcohols in which the solvent mixture of parts (a) and (b) is soluble, and may include water, or a water-compatible solvent mixture.

7. The antifungal compound of claim 6 wherein the lower aliphatic alcohol is selected from the list consisting of ethanol or isopropanol.

8. The antifungal compound of claim 6 wherein the distribution solvent or mixture of solvents has a boiling point less than about 85.degree.C.

9. The antifungal composition of claim 6 wherein the concentration of the antifungal compound is from about 0.5% to about 3% (w/v), the concentration of the aliphatic alcohol is from about 2% to about 25% (v/v), and the concentration of the distribution solvent or mixture of solvents is from about 75% to about 98% (v/v).

10. The antifungal composition of claim 6 wherein the concentration of the antifungal compound is about 1% (w/v), the concentration of the aliphatic alcohol is about 5% (v/v), and the concentration of the distribution solvent or mixture of solvents is about 85% (v/v).

11. The antifungal composition of claim 1 further including a pharmaceutically effective amount of a second antifungal compound selected from the group consisting of miconazole nitrate, tolnaftate, clotrimazole, itraconazole, fluconazole, ketoconazole, econazole, terconazole, butoconazole, sulconazole, ciclopirox olamine, haloprogin, and griseofulvin.

12. The antifungal composition of claim 10 wherein the concentration of the additional antifungal compound is from about 0.05% to about 3% (w/v).

13. A method for the treatment of fungal infections of the skin or nails which comprises the administration of the antifungal composition of claim 1 topically to the skin or nails in an amount therapeutically effective to treat the fungal infection.

14. A method for the treatment of fungal infections of the skin or nails which comprises the administration of the antifungal composition of claim 10 topically to the skin or nails in an amount therapeutically effective to treat the fungal infection.

15. A method for the treatment of fungal infections of the skin or nails which comprises the administration of the antifungal composition of claim 12 topically to the skin or nails in an amount therapeutically effective to treat the fungal infection.

16. A method for the treatment of fungal infections of the skin or nails which comprises the administration of butenafine in a concentration from about 0.5% to about 3% (w/v), benzyl alcohol in a concentration from about 5% to about 25% (v/v), ethanol in a concentration from about 65% to about 85% (v/v), and water or a water-compatible solvent mixture in a concentration of about 10% (v/v).

17. A method for the treatment of fungal infections of the skin or nails which comprises the administration of butenafine in a concentration of about 1% (w/v), benzyl alcohol in a concentration of about 5% (v/v), ethanol in a concentration of about 85% (v/v), and water or a water-compatible solvent mixture in a concentration of about 10% (v/v).

18. The method of claim 17 wherein said composition further comprises a pharmaceutically effective amount of a second antifungal selected from the group consisting of itraconazole, ketoconazole, econazole and griseofulvin.
